(19) United States
(12) Patent Application Publication
(10) Pub. No.: US 2020/0349451 A1
(43) Pub. Date: Nov. 5, 2020

(54) Physical Property Prediction Method and Physical Property Prediction System

(71) Applicant: Semiconductor Energy Laboratory Co., Ltd., Kanagawa-ken (JP)

(72) Inventors: Kunihiko Suzuki, Isehara, Kanagawa (JP); Satoshi SEO, Sagamihara, Kanagawa (JP); Harue OSAKA, Atsugi, Kanagawa (JP); Yoshitaka DOZEN, Atsugi, Kanagawa (JP)

(73) Assignee: Semiconductor Energy Laboratory Co., Ltd., Kanagawa-ken (JP)

(21) Appl. No.: 16/643,094

(22) PCT Filed: Aug. 24, 2018
(86) PCT No.: PCT/IB2018/056409

§ 371 Date: Feb. 28, 2020

(30) Foreign Application Priority Data
Sep. 6, 2017 (JP) 2017-171334

(51) Int. Cl. G06N 3/12; G06N 20/00; G16C 20/70; G16C 20/30

(52) U.S. Cl. CPC G06N 3/126; G16C 20/30; G16C 20/70; G06N 20/00

(57) ABSTRACT

A physical property prediction method that allows anyone to predict a physical property of an organic compound easily and accurately is provided. A physical property prediction system that allows anyone to predict a physical property of an organic compound easily and accurately is provided. Provided are a physical property prediction method including the step of learning a correlation between a molecular structure and a physical property of an organic compound and the step of predicting the target physical property value from the molecular structure of an object substance, and a physical property prediction system. A plurality of kinds of fingerprinting methods are used at the same time as notation methods of the molecular structure of the organic compound.

### TECHNICAL FIELD

One embodiment of the present invention relates to a physical property prediction method and a physical property prediction device for an organic compound.

### BACKGROUND ART

The physical properties of organic compounds had never been known unless the target substance was synthesized and the properties were directly measured. Experts can guess an approximate value of the physical property of an organic compound having a certain molecular structure in these days when data are accumulated because the property is determined by the molecular structure of an organic compound. Prediction can also be made by computation using the first-principle simulation theory or the like in recent years.

In accordance with required characteristics, an organic compound having the corresponding physical property is selected and used in research and development involving organic compounds. Thus, if an organic compound having a required physical property can be accurately predicted and selected from known substances and unknown substances to be used without being actually synthesized, the development speed is expected to be significantly increased.

Not everyone can make the accurate prediction described above and the simulation requires considerable amount of cost and time under the present circumstances. However, since there are very many candidate organic compounds, a method and a system that allow anyone to predict a physical property of the target organic compound easily and quickly have been desired.

In recent years, a method of classification, estimation, prediction, or the like employing a method of machine learning or the like has advanced significantly. In particular, selection and prediction by deep learning using a convolutional neural network have significantly improved in performance, and produced excellent effects in various fields. However, in the field covering organic compounds, there are as yet almost no sufficient methods of describing organic compounds that allow, with an adequate information volume, computers to understand a structure without any failure and to accurately extract a feature related to a physical property. Thus, the physical property prediction method and system that allow anyone to predict a physical property of an organic compound easily and accurately have not been achieved yet.

Patent Document 1 discloses a novel substance searching method using machine learning and a device thereof.

### PRIOR ART DOCUMENT

### Patent Document

[Patent Document 1] Japanese Published Patent Application No. 2017-91526

### SUMMARY OF THE INVENTION

### Problems to be Solved by the Invention

An object of one embodiment of the present invention is to provide a physical property prediction method that allows anyone to predict a physical property of an unknown organic compound easily and accurately. Another object is to provide a physical property prediction system that allows anyone to predict a physical property of an organic compound easily and accurately.

### Means for Solving the Problems

One embodiment of the present invention is a method of predicting a physical property of an organic compound, which includes the step of learning a correlation between a molecular structure and a physical property of an organic compound and the step of predicting a target physical property from a molecular structure of an object substance on the basis of a result of the learning. A plurality of kinds of fingerprinting methods are used at the same time as notation methods of the molecular structure of the organic compound.

Another embodiment of the present invention is a method of predicting a physical property of an organic compound, which includes the step of learning a correlation between a molecular structure and a physical property of an organic compound and the step of predicting a target physical property from a molecular structure of an object substance on the basis of a result of the learning. Two kinds of fingerprinting methods are used at the same time as notation methods of the molecular structure of the organic compound.

Another embodiment of the present invention is a method of predicting a physical property of an organic compound, which includes the step of learning a correlation between a molecular structure and a physical property of an organic compound and the step of predicting a target physical property from a molecular structure of an object substance on the basis of a result of the learning. Three kinds of fingerprinting methods are used at the same time as notation methods of the molecular structure of the organic compound.

Another embodiment of the present invention is the method of predicting a physical property in the above structure, in which the fingerprinting methods include at least any one of an Atom pair type, a Circular type, a Substructure key type, and a Path-based type.

Another embodiment of the present invention is the method of predicting a physical property in the above structure, in which the plurality of fingerprinting methods are selected from an Atom pair type, a Circular type, a Substructure key type, and a Path-based type.

Another embodiment of the present invention is the method of predicting a physical property in the above structure, in which the fingerprinting methods include an Atom pair type and a Circular type.

Another embodiment of the present invention is the method of predicting a physical property in the above structure, in which the fingerprinting methods include a Circular type and a Substructure key type.

Another embodiment of the present invention is the method of predicting a physical property in the above structure, in which the fingerprinting methods include a Circular type and a Path-based type.

Another embodiment of the present invention is the method of predicting a physical property in the above structure, in which the fingerprinting methods include an Atom pair type and a Substructure key type.

Another embodiment of the present invention is the method of predicting a physical property in the above structure, in which the fingerprinting methods include an Atom pair type and a Path-based type.

Another embodiment of the present invention is the method of predicting a physical property in the above structure, in which the fingerprinting methods include an Atom pair type, a Substructure key type, and a Circular type.

Another embodiment of the present invention is the method of predicting a physical property in the above structure, in which r is greater than or equal to 3 when the Circular type is used for the fingerprinting methods.

Another embodiment of the present invention is the method of predicting a physical property in the above structure, in which r is greater than or equal to 5 in the fingerprinting method of the Circular type.

Another embodiment of the present invention is the method of predicting a physical property in the above structure, in which notations of all organic compounds are different when molecular structures of the organic compounds to be learned are notated using at least one of the fingerprinting methods.

Another embodiment of the present invention is the method of predicting a physical property in the above structure, in which at least one of the fingerprinting methods is capable of expressing information about a structure featuring a physical property to be predicted.

Another embodiment of the present invention is the method of predicting a physical property in the above structure, in which at least one of the fingerprinting methods is capable of expressing at least one of a substituent, a substitution position of the substituent, a functional group, the number of elements, kinds of elements, valences of elements, a bond order, and an atomic coordinate.

Another embodiment of the present invention is the method of predicting a physical property in the above structure, in which the physical property is any one or more of an emission spectrum; a half width; emission energy; an excitation spectrum; an absorption spectrum; a transmission spectrum; a reflectance spectrum; a molar absorption coefficient; excitation energy; a transient emission lifetime; a transient absorption lifetime; an S1 level; a T1 level; an Sn level; a Tn level; a Stokes shift value; an emission quantum yield; oscillator strength; an oxidation potential; a reduction potential; a HOMO level; a LUMO level; a glass transition point; a melting point; a crystallization temperature; a decomposition temperature; a boiling point; a sublimation temperature; carrier mobility; a refractive index; an orientation parameter; a mass-to-charge ratio; a spectrum, a chemical shift and the number of the elements, or a coupling constant in an NMR measurement; and a spectrum, a g-factor, a D value, or an E value in an ESR measurement.

Another embodiment of the present invention is a system of predicting a physical property of an organic compound, which includes an input means, a data server, a learning means that learns a correlation between a molecular structure and a physical property of an organic compound stored in the data server, a prediction means that predicts a target physical property on the basis of a result of the learning from a molecular structure of an object substance input from the input means, and an output means that outputs a predicted physical property value. A plurality of kinds of fingerprinting methods are used at the same time as notation methods of the molecular structure of the organic compound.

Another embodiment of the present invention is a system of predicting a physical property of an organic compound, which includes an input means, a data server, a learning means that learns a correlation between a molecular structure and a physical property of an organic compound stored in the data server, a prediction means that predicts a target physical property on the basis of a result of the learning from a molecular structure of an object substance input from the input means, and an output means that outputs a predicted physical property value. Two kinds of fingerprinting methods are used at the same time as notation methods of the molecular structure of the organic compound.

Another embodiment of the present invention is a system of predicting a physical property of predicting a physical property of an organic compound, which includes an input means, a data server, a learning means that learns a correlation between a molecular structure and a physical property of an organic compound stored in the data server, a prediction means that predicts a target physical property on the basis of the result of the learning from a molecular structure of an object substance input from the input means, and an output means that outputs a predicted physical property value. Three kinds of fingerprinting methods are used at the same time as notation methods of the molecular structure of the organic compound.

Another embodiment of the present invention is the system of predicting a physical property in the above structure, in which the fingerprinting methods include at least any one of an Atom pair type, a Circular type, a Substructure key type, and a Path-based type.

Another embodiment of the present invention is the system of predicting a physical property in the above structure, in which the plurality of fingerprinting methods are selected from an Atom pair type, a Circular type, a Substructure key type, and a Path-based type.

Another embodiment of the present invention is the system of predicting a physical property in the above structure, in which the fingerprinting methods include an Atom pair type and a Circular type.

Another embodiment of the present invention is the system of predicting a physical property in the above structure, in which the fingerprinting methods include a Circular type and a Substructure key type.

Another embodiment of the present invention is the system of predicting a physical property in the above structure, in which the fingerprinting methods include a Circular type and a Path-based type.

Another embodiment of the present invention is the system of predicting a physical property in the above structure, in which the fingerprinting methods include an Atom pair type and a Substructure key type.

Another embodiment of the present invention is the system of predicting a physical property in the above structure, in which the fingerprinting methods include an Atom pair type and a Path-based type.

Another embodiment of the present invention is the system of predicting a physical property in the above structure, in which the fingerprinting methods include an Atom pair type, a Substructure key type, and a Circular type.

Another embodiment of the present invention is the system of predicting a physical property in the above structure, in which r is greater than or equal to 3 when the Circular type is used for the fingerprinting method.

Another embodiment of the present invention is the system of predicting a physical property in the above structure, in which r is greater than or equal to 5 in the fingerprinting method of the Circular type.

Another embodiment of the present invention is the system of predicting a physical property in the above structure, in which notations of all organic compounds are different when molecular structures of the organic compounds to be learned are notated using at least one of the fingerprinting methods.

Another embodiment of the present invention is the system of predicting a physical property in the above structure, in which at least one of the fingerprinting methods is capable of expressing information about a structure featuring a physical property to be predicted.

Another embodiment of the present invention is the system of predicting a physical property in the above structure, in which at least one of the fingerprinting methods is capable of expressing at least one of a substituent, a substitution position of the substituent, a functional group, the number of elements, kinds of elements, valences of elements, a bond order, and an atomic coordinate.

Another embodiment of the present invention system of predicting a physical property, in which a physical property is any one or more of an emission spectrum; a half width; emission energy; an excitation spectrum; an absorption spectrum; a transmission spectrum; a reflectance spectrum; a molar absorption coefficient; excitation energy; a transient emission lifetime; transient absorption lifetime; an S1 level; a T1 level; an Sn level; a Tn level; a Stokes shift value; an emission quantum yield; oscillator strength; an oxidation potential; a reduction potential; a HOMO level; a LUMO level; a glass transition point; a melting point; a crystallization temperature; a decomposition temperature; a boiling point; a sublimation temperature; carrier mobility; a refractive index; an orientation parameter; a mass-to-charge ratio; a spectrum, a chemical shift and the number of the elements, or a coupling constant in an NMR measurement; and a spectrum, a g-factor, a D value, or an E value in an ESR measurement.

### Effect of the Invention

According to one embodiment of the present invention, a physical property prediction method that allows anyone to predict a physical property of an unknown organic compound easily and accurately can be provided. A physical property prediction system that allows anyone to predict a physical property of an organic compound easily and accurately can be provided.

### BRIEF DESCRIPTION OF THE DRAWINGS

FIG. 1 A flow chart showing one embodiment of the present invention.

FIG. 2 A diagram showing a method of converting a molecular structure by a fingerprinting method.

FIG. 3 A diagram illustrating kinds of fingerprinting methods.

FIG. 4 A diagram illustrating conversion from the SMILES notation to a notation by a fingerprinting method.

FIG. 5 A diagram illustrating kinds of fingerprinting methods and duplicating notations.

FIG. 6 A diagram illustrating examples in which a molecular structure is notated by a plurality of fingerprinting methods.

FIG. 7 A diagram illustrating a structure of a neural network.

FIG. 8 A diagram showing a physical property prediction system of one embodiment of the present invention.

FIG. 9 Diagrams illustrating a configuration of a neural network.

FIG. 10 A diagram illustrating a configuration example of a semiconductor device having a function of performing an operation.

FIG. 11 A diagram illustrating a specific configuration example of memory cells.

FIG. 12 A diagram illustrating a configuration example of an offset circuit OFST.

FIG. 13 A diagram showing a timing chart of an operation example of a semiconductor device.

FIG. 14 Diagrams showing results of physical property prediction.

### MODE FOR CARRYING OUT THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the descriptions in the following embodiments.

### Embodiment 1

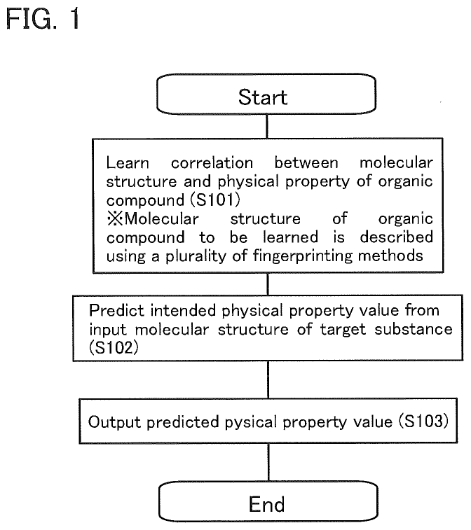

A physical property prediction method of one embodiment of the present invention can be shown by a flow chart like FIG. 1, for example. In the physical property prediction method of one embodiment of the present invention, a correlation between a molecular structure and a physical property of an organic compound is first learned as in FIG. 1 (S101).

At this time, the molecular structure needs to be described by a mathematical expression in order that machine learning of the correlation between the molecular structure and the physical property can be performed. For mathematization of molecular structures, RDKit, which is an open-source cheminformatics toolkit, can be used. In the RDKit, the SMILES notation (Simplified molecular input line entry specification syntax) of the input molecular structure can be converted into mathematical expression data by a fingerprinting method.

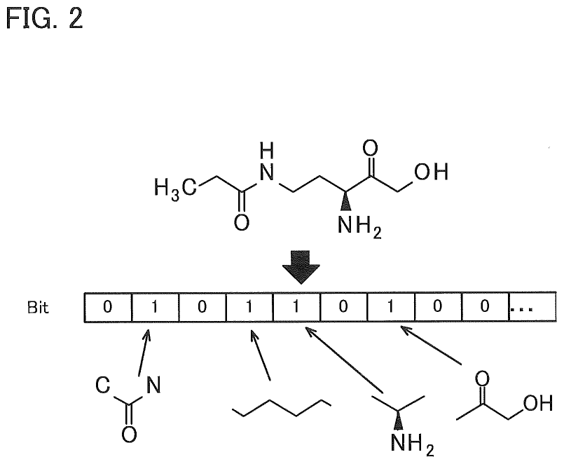

In a fingerprinting method, as illustrated in FIG. 2, for example, substructures (fragments) of a molecular structure are assigned to the respective bits to represent the molecular structure; “1” is set to the bit if the corresponding substructure is present in the molecule and “0” is set to the bit if the corresponding substructure is absent. That is, the fingerprinting method can provide a mathematical expression by extracting features of a molecular structure. In general, in an expression of a molecular structure expressed by a fingerprinting method, the bit length is several hundreds to several tens of thousands, which is a size easy to handle. Since a molecular structure is represented by a mathematical expression of 0 and 1, the use of a fingerprinting method enables significantly high-speed calculation processing.

There are many kinds of fingerprinting methods (methods considering the difference in bit generation algorithm, atom types or bond types, or conditions of aromaticity, methods dynamically generating a bit length using a hash function, or the like), which have different features.

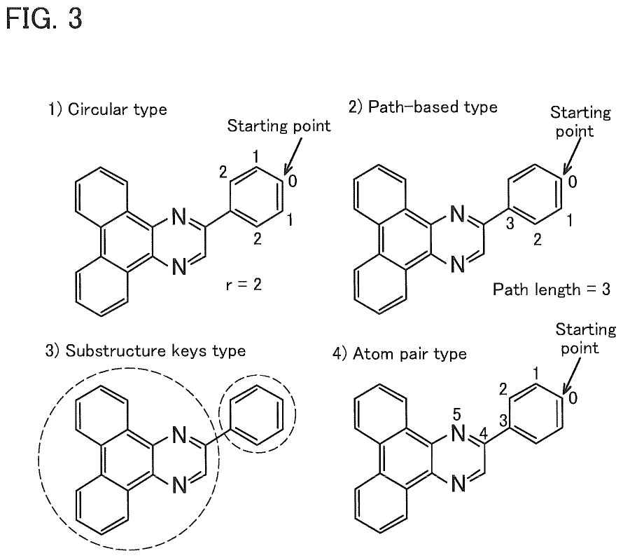

As illustrated in FIG. 3, the following are typical kinds of fingerprinting methods: 1) Circular type (neighboring atoms within a specified radius from the atom as a starting point are considered a substructure); 2) Path-based type (atoms at a specified length of path (Path length) from the atom as a starting point are considered a substructure); 3) Substructure keys type (a substructure is defined for each bit); 4) Atom pair type (atom pairs generated for all the atoms in a molecule are considered a substructure); and the like. The RDKit is equipped with these various types of fingerprints.

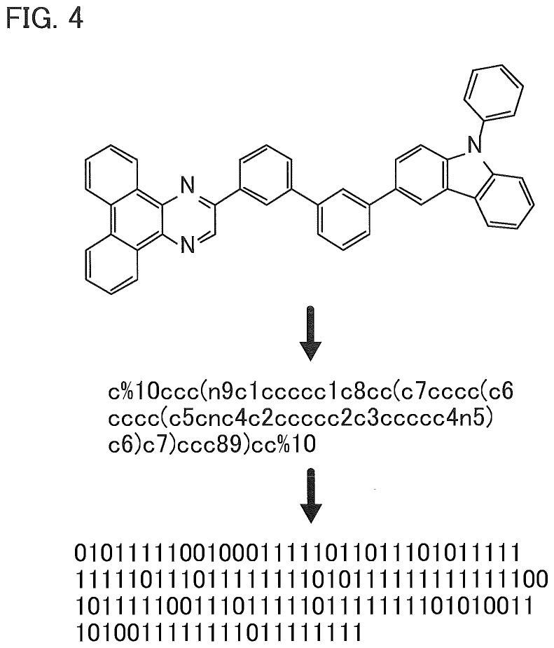

FIG. 4 is an example in which the molecular structure of a certain organic compound is actually represented as a mathematical expression by a fingerprinting method. In this manner, the molecular structure is once converted into the SMILES notation and then can be converted into a fingerprint.

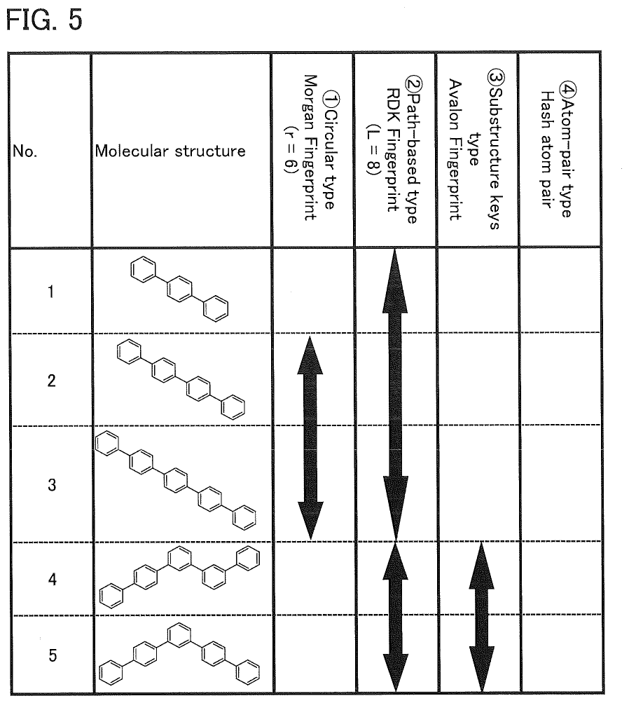

When molecular structures of organic compounds are expressed by a fingerprinting method, different organic compounds having similar structures are represented by the same mathematical expression in some cases. Although there are some kinds of fingerprinting methods differing in notation methods as described above, the tendency of the compounds that become the same differs among notation methods as shown in [1] Circular type (Morgan Fingerprint), [2] Path-based type (RDK Fingerprint), [3] Substructure keys type (Avalon Fingerprint), and [4] Atom pair type (Hash atom pair) in FIG. 5. In FIG. 5, molecules within the corresponding double-headed arrow show the same mathematical expression (notation). As at least one of fingerprinting methods for learning, a method with which notations of organic compounds for learning are all different when the molecular structures of the organic compounds are notated is preferably used. FIG. 5 reveals that different compounds can be notated without duplicating notations by the Atom pair type. However, depending on the population of organic compounds to be learned, notation without duplicating notations can also be possible by another notation methods in some cases.

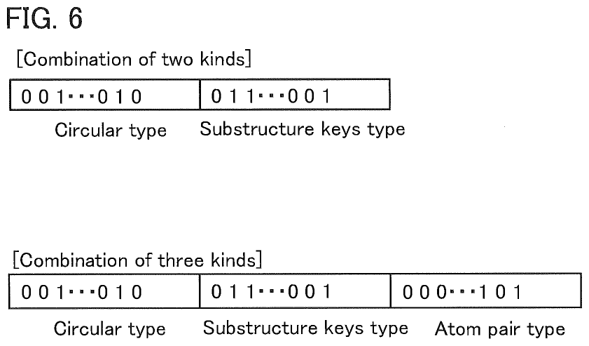

Here, one embodiment of the present invention is characterized in that a plurality of different kinds of fingerprinting methods are used when organic compounds to be learned are notated by fingerprinting methods. Although any number of kinds may be used, two or three kinds or so are manageable in terms of the volume of data and preferred. When learning is performed by a plurality of kinds of fingerprinting methods, a mathematical expression notated by one kind of fingerprinting method which is connected to the following mathematical expression notated by another kind of fingerprinting method may be used, or the presence of a plurality of kinds of different mathematical expressions for one organic compound may be assumed for the learning. FIG. 6 shows examples of methods in which a plurality of fingerprints of different types are used to describe a molecular structure.

A fingerprint is a method of describing the presence or absence of a substructure, in which information about the whole molecular structure is lost. However, when a molecular structure is mathematized using a plurality of fingerprints of different types, different substructures are generated by the respective types of fingerprints, so that information about the presence or absence of these substructures can complement information about the whole molecular structure. In the case where a feature that cannot be sufficiently expressed by a certain fingerprint affects a physical property value significantly or in the case where the feature affects a difference between physical property values of some compounds, the method of describing a molecular structure using a plurality of fingerprints of different types is effective because the feature is complemented by another fingerprint.

The Atom pair type and the Circular type are preferably used for notation by two kinds of fingerprinting methods, because physical property prediction can be accurately performed in this structure.

The Atom pair type, the Circular type, and the Substructure keys type are preferably used for notation by three kinds of fingerprinting methods, because physical property prediction can be accurately performed in this structure.

In the case where a Circular type fingerprinting method is used, a radius r is preferably greater than or equal to 3, further preferably greater than or equal to 5. The term radius r is the number of bonded elements counted starting from a certain element as 0.

When a fingerprinting method to be used is selected, at least one with which notations of organic compounds for learning are all different when molecular structures of the organic compounds are notated is preferably selected as described above.

Although an increase in bit length (the number of bits) to be expressed can reduce the possibility of generating the notations of organic compounds for learning which agree exactly with each other, a fingerprint has a trade-off problem that an excessive increase in bit length increases the calculation cost or the database management cost. When a plurality of fingerprints are used at the same time for expression, the different fingerprint types in combination might avoid an exact agreement between notations of a plurality of molecular structures as a whole even if the notations agree exactly with each other according to one fingerprint type. This can allow the shortest possible bit length to create a state where no organic compounds have exactly the same notations by fingerprints. Furthermore, features of a molecular structure can be extracted by a plurality of methods, and accordingly learning efficiency is high and over-learning is unlikely to occur. There is no particular limitation on the bit lengths of fingerprints to be generated. However, in consideration of the calculation cost or the database management cost, when molecules each have a molecular weight up to approximately 2000 and the bit length for each fingerprint type is 4096 or less, preferably 2048 or less, or 1024 or less depending on circumstances, a state where the fingerprints of the molecules agree exactly with each other can be avoided, and fingerprints with high learning efficiency can be generated.

The bit lengths of fingerprints generated by the respective fingerprint types are adjusted as appropriate in consideration of features of the types or the whole molecular structure to be learned, and not necessarily the same. For example, the bit lengths may be represented as 1024 bits in the Atom pair type and 2048 bits in the Circular type, and they may be connected to each other.

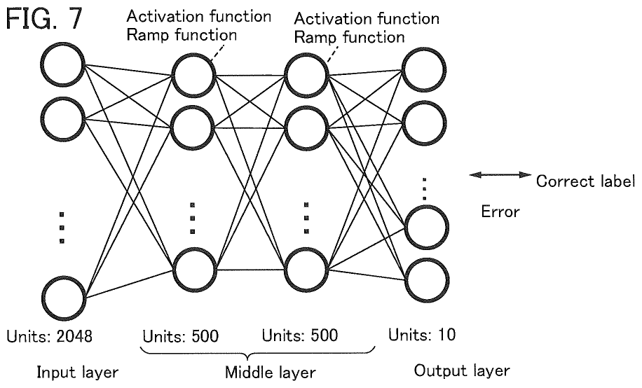

Although any method may be used for machine learning, a neural network is preferably used. Learning by a neural network is performed by constructing a structure as in FIG. 7, for example. For example, Python can be used as a programming language and Chainer or the like can be used as a framework of machine learning. For evaluation of the validity of a prediction model, some of data on a physical property value are used for testing and the rest is used for learning.

Examples of a physical property value to be learned in connection with a molecular structure are an emission spectrum; a half width; emission energy; an excitation spectrum; an absorption spectrum; a transmission spectrum; a reflectance spectrum; a molar absorption coefficient; excitation energy; a transient emission lifetime; a transient absorption lifetime; an S1 level; a T1 level; an Sn level; a Tn level; a Stokes shift value; an emission quantum yield; oscillator strength; an oxidation potential; a reduction potential; a HOMO level; a LUMO level; a glass transition point; a melting point; a crystallization temperature; a decomposition temperature; a boiling point; a sublimation temperature; carrier mobility; a refractive index; orientation parameters; a mass-to-charge ratio; a spectrum, a chemical shift and the number of the elements, or a coupling constant in an NMR measurement; a spectrum, a g-factor, a D value, or an E value in an ESR measurement, and the like.

These may be obtained by measurement or may be obtained by simulation. A measurement object is selected as appropriate from a solution, a thin film, powder, and the like. Note that physical property values obtained in the same unit under the same measurement conditions or simulation conditions are preferably learned. If the conditions cannot be uniform, it is preferable that, for some (at least two kinds of compounds or more, preferably 1% or more, further preferably 3% or more) of learning data, physical property values of the same compound be measured or simulated under each measurement condition so that the correlation between the values obtained by the measurements or simulations under different conditions can be learned. In addition, information about the conditions themselves are preferably incorporated into the learning data at the same time.

One or a plurality of kinds of physical property values may be learned and predicted. When there is a correlation between physical property values, the plurality of kinds of physical property values are preferably learned at a time because learning efficiency increases and prediction accuracy increases. Such learning is preferred and efficient also when there is no or low correlation between the physical property values because a plurality of physical property values can be predicted at a time.

Physical property values determined based on the same or similar characteristics are given as the physical property values that are effective when learned in combination. For example, physical property values belonging to physical property values related to optical characteristics, physical property values related to chemical characteristics or electric characteristics, and the like are combined as appropriate and learned. As the physical property values related to optical characteristics, an absorption peak, an absorption edge, a molar absorption coefficient, an emission peak, a half width of an emission spectrum, an emission quantum yield, and the like are given. Examples are an emission peak of a solution and an emission peak of a thin film, an emission peak measured at room temperature and an emission peak measured at a low temperature, the S1 level (lowest singlet excited level), T1 level (lowest triplet excited level), Sn level (higher singlet excited level), and Tn level (higher triplet excited level) that are obtained by simulation, and the like. Two or more selected from the above are preferably learned in combination.

Although physical property values to be learned and predicted are selected as appropriate, physical property values obtained by a measurement method or simulation described below, for example, are preferably selected for an organic EL device. The physical property values are each described.

As for an emission spectrum, the emission intensity in a certain fixed wavelength range per wavelength is determined and used as values for learning. The values may be absolute values; however, the highest local maximum value is preferably normalized for prediction of the spectrum. When a comparison between absolute values is intended, the maximum intensity, an emission quantum yield, or the like is described in parallel as appropriate.

Measurement is performed in a state of a solution, a thin film, powder, or the like. The value of a solution is preferred for prediction of the emission color of a dopant in an organic EL device. At this time, measurement is preferably performed in a solvent whose polarity is as close to that of a host used in an actual device as possible (a difference in dielectric constant between the solvent and the actual device is preferably within 10, preferably approximately an absolute value of 5). The solvent is preferably toluene, chloroform, dichloromethane, or the like, for example. In the case of a solution, the concentration is preferably approximately 10⁻⁴ to 10⁻⁶ M so that no intermolecular interaction occurs. A thin film in which organic matter such as a host is subjected to doping is also preferred for prediction of the emission color of a dopant. In this case, the doping concentration is preferably similar to that in the device, preferably approximately 0.5 w % to 30 w %. As an emission spectrum, there is a fluorescence spectrum or a phosphorescence spectrum. A phosphorescence spectrum of an iridium complex or the like using a heavy atom can be measured at room temperature when the iridium complex or the like is brought into a deoxidized state. Otherwise, measurement can be performed at low temperature (100 K to 10 K) set with liquid nitrogen, liquid helium, or the like. Note that the spectrum can be measured with a fluorescence spectrophotometer. The term half width refers to a spectrum width when emission intensity becomes a half of the intensity of the local maximum value.

As emission energy, a value that meets the purpose is learned. In the case where there are a plurality of local maximum values, the maximum intensity value is preferably found for the prediction of the emission color of a dopant in an organic EL device, for example. As energy of a host material, a carrier-transport layer, or the like, the local maximum value on the shortest wavelength side or the value of the rising portion on the short wavelength side (the value of the intersection between the base line and the tangent in a plot of 70 to 50% of the local maximum value of the intensity on the shortest wavelength side) may be used. The value may be found by formation of a tangent at a point where the derivative of the rising portion on the short wavelength side is maximum.

As for an absorption spectrum, a transmission spectrum, and a reflectance spectrum, the absorbance, absorbance, transmittance, and reflectivity in a certain fixed wavelength range per wavelength are determined and used as values for learning. Absolute values or normalized values are learned depending on the purposes; values normalized at a given wavelength are learned when a comparison between spectrum shapes is intended. Absolute values are learned as they are when a comparison between the absolute values is intended. When conditions such as concentrations or thicknesses are not made uniform, such conditions are preferably described in parallel with the absolute values of intensity. For example, when the influence of light extraction efficiency or the like in an organic EL device is intended to be predicted, the transmittance and thickness of a thin film are preferably learned in parallel. For example, when the efficiency of energy transfer from a host to a dopant in an organic EL device is intended to be predicted, the molar absorption coefficient of the dopant is preferably used as the intensity. Note that the spectra can be measured with an absorptiometer.

Excited energy can be found from an absorption spectrum. The wavelength of an absorption edge and the wavelength at local maximum absorbance, the intensity at such a wavelength, the intensity at a given wavelength, or the like is learned as appropriate. An absorption edge is found from the value of the intersection between the base line and the tangent in a plot of 70 to 50% of the local maximum absorption value of the intensity on the longest wavelength side, for example. A tangent may be formed at a point of a curve showing absorption decay from the local maximum absorption on the longest wavelength side, where the (negative) derivative of the curve is minimum.

A Stokes shift value can be found from a difference between the maximum excitation wavelength and the maximum emission wavelength. It may be a difference between the maximum absorption wavelength and the maximum emission wavelength. In the case of a light-emitting material, for example, a Stokes shift value is preferably learned as energy (eV). The smaller this value is, the smaller structure relaxation is regarded, which indicates that the emission quantum yield is high.

The transient emission lifetime can be found from the time during which the emission intensity decays (lifetime) by irradiation of a sample with pulsed light. At this time, the emission intensity at each time in a certain time range and the value of the lifetime determined from the emission intensity are learned as appropriate. In the case of a waveform, values are preferably normalized. The initial integrated intensity at every wavelength may be normalized and the intensity at each wavelength may be a relative value. For example, in the case of a light-emitting material, the emission quantum yield is considered high as it decays rapidly (as the lifetime is shorter). Note that this can be measured with a fluorescence (luminescence) lifetime measurement apparatus. In the case where the transient emission lifetime of a light-emitting device is measured, not the photoexcitation but electrical excitation may be performed. In other words, a pulsed voltage may be applied to a light-emitting device, and the time during which the emission intensity decays (lifetime) may be measured. Note that the time required for the emission intensity to reach 1/e is usually used as an indicator of the time during which the emission intensity decays (lifetime).

An S1 level can be found from an absorption edge or the local maximum value on the long wavelength side of an absorption spectrum, the highest local maximum value of an excitation spectrum, or the highest local maximum value or the value of the rising portion on the short wavelength side of an emission spectrum. A T1 level can be found from an absorption edge or the local maximum value on the long wavelength side of an absorption spectrum found by transient absorption measurement or the like, the highest local maximum value of a phosphorescence spectrum, or a peak wavelength on the short wavelength side or the value of the rising portion on the short wavelength side of a phosphorescence spectrum. Note that a way of finding an absorption edge or the value of the rising portion of an emission spectrum is as described above. An S1 level and a T1 level can also be found by simulation. For example, the levels can be found as excited energy by time-dependent density functional theory after a ground state (S0) is structurally optimized with a density functional theory such as Gaussian, which is a quantum chemistry computational program. In a similar manner, an Sn level (a singlet level above S1) and a In level (a triplet level above T1) can be found. Here, as transition probability, oscillator strength may be found at the same time. For example, in the case of a light-emitting material, the oscillator strength is preferably high, in which case light is probably easily emitted at the level. A difference between potential energy of structure-optimized S0 and potential energy of structure-optimized T1 found by a density functional theory may be regarded as a T1 level.

An emission quantum yield can be found with an absolute quantum yield measurement apparatus.

An oxidation potential and a reduction potential can be measured by cyclic voltammetry (CV). A HOMO level and a LUMO level can also be found by CV measurement using the oxidation-reduction potential of a standard sample (e.g., ferrocene) with known redox potential energy (eV) as a reference. However, a HOMO level can also be measured in a solid (thin film or powder) state by photoelectron spectroscopy in air (PESA). In this case, the LUMO can be found in such a manner that a band gap is found from an absorption edge of an absorption spectrum and the energy value is added to the HOMO level found by PESA. For example, in order that emission energy be evaluated when an exciplex is generated between two molecules in an organic EL device, an energy difference between the molecules, one molecule whose HOMO level is higher (HOMO level is shallower) and the other whose LUMO level is lower (LUMO level is deeper), is found. In this case, a HOMO level and a LUMO level found by CV are preferably used. By a density functional theory such as Gaussian, which is a quantum chemistry computational program, a HOMO level, a LUMO level, a HOMO-n level (level of an occupied orbit below the HOMO), and a LUMO+n (level of an unoccupied orbit above the LUMO) can be found.

The glass transition point, the melting point, and the crystallization temperature can be found with a differential scanning calorimetry (DSC) apparatus. The temperature rising rate is preferably set to be constant at 10 to 50° C./min for measurement. The decomposition temperature, the boiling point, and the sublimation temperature can be found with a thermogravimetry-differential thermal analysis (TG-DTA) apparatus. The results of measurement under an atmospheric pressure or a reduced pressure are used as appropriate. The value by measurement under reduced pressure can be referred to for the sublimation purification temperature or the evaporation temperature, and the value obtained when the weight is reduced by approximately 5 to 20% is preferably used. The temperature rising rate is preferably set to be constant at 10 to 50° C./min for measurement.

Carrier mobility can be found by a time-of-flight (TOF) method utilizing transient photocurrent. In the TOF method, a sample film is sandwiched between electrodes, carrier is generated by pulse photoexcitation in a state where a DC voltage is applied, and mobility is estimated from the traveling time of the generated carrier (transit response of current). In this case, the film thickness is preferably 3 μm or more. In another method, when current-voltage characteristics of the sample film follow space-charge-limited current (SCLC), the current-voltage characteristics are fitted with the formula of SCLC so that the mobility can be found. A method of finding the mobility from the frequency dependent characteristics of conductance or capacitance obtained by impedance spectrometry is also reported. The mobility at a certain voltage (electric field intensity) can be found by any method, and can be utilized as a physical property value. The electric field intensity dependence of the mobility is plotted and extrapolation is performed, whereby mobility μ₀ when there is no electric field can be found and this may be utilized as a physical property value.

A refractive index and orientation parameters can be found with a spectroscopic ellipsometry apparatus. For example, in the case of an organic EL device, the refractive index in the visible region is preferably low, in which case light extraction efficiency is improved. There are some examples of reports on orientation parameters, and an orientation parameter S, for example, is often used in the case of an organic EL device. The orientation parameter S can be calculated by measurement of light absorption anisotropy by spectroscopic ellipsometry. For a fluorescent substance, S is preferably close to −0.5 at a wavelength corresponding to absorption derived from the lowest singlet excited state (S1), in which case the transition dipole moment is probably more horizontal to the light-extraction surface of a substrate or the like and light extraction efficiency is increased. For a phosphorescent substance, the focus is placed on absorption in the lowest triplet excited state (T1). The orientation is random when S is 0 and the orientation is perpendicular when S is 1. As another orientation parameter, the proportion of a perpendicular component when the transition dipole moment is divided into a component horizontal to a substrate and a component perpendicular to the substrate may be used. This parameter can be found in such a manner that angle dependence of the p-polarization intensity of photoluminescence (PL) or electroluminescence (EL) is examined and fitted.

As for a mass-to-charge ratio (m/z), the detected intensity in a certain fixed range of mass-to-charge ratio numbers per unit is determined and used as a value for learning. Absolute values or normalized values are learned depending on the purposes; values normalized at a given wavelength, such as the m/z of a parent ion, are learned when a comparison between spectrum shapes is intended. Absolute values are learned as they are when a comparison between the absolute values is intended. The m/z can be measured with a mass spectrometry apparatus. As ionization methods, there are an electron ionization method, a chemical ionization method, an electrolysis dissociation method, a fast atom bombardment method, matrix-assisted laser desorption/ionization method, an electrospray ionization method, an atmospheric pressure chemical ionization method, an inductively coupled plasma method, and the like can be given. At this time, a fragment (a daughter ion) due to decomposition (bond dissociation) of a molecule (a parent molecule) might be detected at the same time, and the detected m/z and the detected intensity ratio to the parent ion indicate a feature of the molecule. For example, fragments with the same m/z might be detected from the molecules having the same substituents. Therefore, when the m/z of the parent ion and the fragment and the detected intensity ratio therebetween are learned, the m/z of a fragment of other compounds or the detected intensity ratio to the parent ion, or the like, can be predicted. In general, the stronger the ionization energy is, the higher the generation proportion of a fragment becomes.

As for an NMR (nuclear magnetic resonance) spectrum, the signal intensity in a certain fixed chemical shift range per chemical shift value is determined used as a value for learning. A chemical shift value of a peak, an integral value of the intensity (the number of elements), a J value (a coupling constant), or the like may be described in parallel. In that case, description is preferably made such that the sum of the integral values of the molecule is the number of elements of measured elements. Note that NMR measurement can analyze the molecular structure of a substance at an atomic level. The molecules having the same substituents tend to exhibit similar spectra at a similar chemical shift value, for example. The spectrum can be measured with an NMR apparatus.

As for an ESR (electron spin resonance) spectrum, the detected intensity in a certain fixed magnetic field intensity range, magnetic flux density (tesla) range, and rotation angle per unit is determined and learned as a value. The value may be described as a g-value (g-factor), the square of a g-value, the volume of spins, the spin density, or the like. In ESR measurement, a resonance phenomenon of a sample including an unpaired electron due to microwave absorption involved with transition of a spin of the unpaired electron in a magnetic field is observed. Thus, ESR is effective for the measurement of a paramagnetic substance having an unpaired electron. Since ESR can be used to observe a triplet state, information about a spin state at a triplet excited state can be obtained when ESR measurement is performed, for example, at low temperature (100 K to 10 K) while irradiated with excitation light is performed. At this time, this may be described as a D value (at the magnitude of interaction between two electron spins) or an E value (the magnitude representing how the orbit of an electron is deviated from axisymmetry). The spectrum can be measured with an ESR apparatus.

When the learning step ends, the object physical property value is then predicted from the input molecular structure of the target substance on the basis of the learned results (S102).

Lastly, the predicted physical property value is output (S103).

As described above, one embodiment of the present invention is a method of predicting a physical property of an organic compound, by which a variety of physical property values can be predicted and the prediction can be made more accurate since the method uses a plurality of fingerprints for learning of the molecular structure of the organic compound.

### Embodiment 2

In Embodiment 2, a system of prediction of a physical property of an organic compound, which is one embodiment of the present invention, is described.

### <Structure Example>

A physical property prediction system 10 of one embodiment of the present invention includes at least an input means, a learning means, a prediction means, an output means, and a data server. As long as these can transmit data to each other, they may be integrated into one device, may be different devices, or may partly be integrated into one device, or the data server may be a cloud. They are collectively referred to as a physical property prediction system.

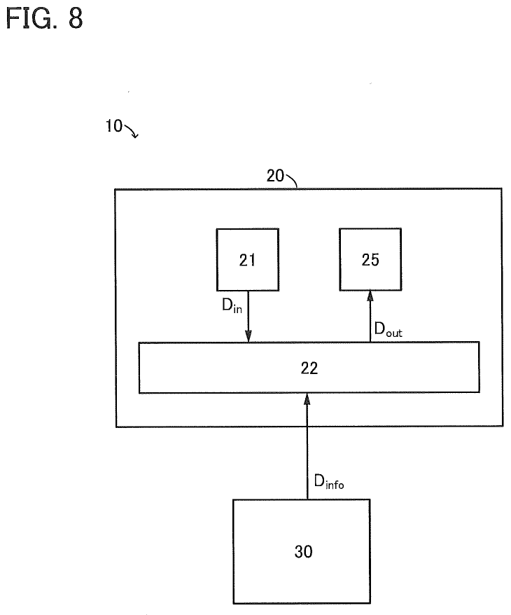

Referring to FIG. 8, description is made as one embodiment of the present invention using an example in which a physical property prediction system composed of an information terminal including an input means, a learning means, a prediction means, and an output means and a data server. An information terminal 20 includes an input portion, the learning means, the prediction means, and an output portion and is capable of transmitting data with the data server provided separately.

The information terminal 20 includes an input portion 21, an arithmetic portion 22, and an output portion 25 as main structures. The arithmetic portion 22 serves as the learning means and the prediction means at the same time. The arithmetic portion 22 preferably includes a neural network circuit. Data offered by the data server becomes data to be learned or predicted by a neural network circuit 26. When part of the data is used as verification data and teacher data for the learning means that has done learning, a weight coefficient in the neural network circuit can be updated and a learned weight coefficient can be generated. This can further improve the accuracy of prediction.

In FIG. 8, a signal flow from the input portion 21, the arithmetic portion 22, a data server 30, and the output portion 25 in this order is indicated by arrows. In this specification, a signal can be replaced with data or information as appropriate.

The data server 30 provides the learning means in the arithmetic portion 22 with the structure and physical property value of an organic compound which are to be learned. The structure of an organic compound to be provided is notated using two or more kinds of fingerprints. The learning means of the arithmetic portion 22 preferably includes the neural network circuit.

The input portion 21 has a function of enabling a user to input information. Specific examples of the input portion 21 include all input means such as a keyboard, a mouse, a touch panel, a pen tablet, a microphone, and a camera.

Input information Dᵢₙ is data to be output from the input portion 21 to the arithmetic portion 22. The input information Dᵢₙ is information input by a user. For example, in the case where the input portion 21 is a touch panel, the input information Dᵢₙ is information obtained by text input with the touch panel operation. Alternatively, in the case where the input portion 21 is a microphone, the input information Dᵢₙ is information obtained by sound input by a user. Alternatively, in the case where the input portion 21 is a camera, the input information Dᵢₙ is information obtained by image processing of imaging data.

The input information Dᵢₙ is information relating to the structure of an organic compound whose physical property is intended to be predicted. When a structural formula, an image of the structure, a substance name, and the like which are not fingerprint notations are input, such information is input to the prediction means in the arithmetic portion 22 after passing through a conversion means as appropriate. The prediction means predicts the physical property of an input organic compound on the basis of results learned in advance by the learning means.

The prediction result is output through the output portion.

In the case where the arithmetic portion includes the neural network circuit, the neural network circuit preferably includes a product-sum arithmetic circuit capable of executing product-sum arithmetic processing. The product-sum arithmetic circuit preferably includes a memory circuit for storing weight data. A memory element included in the memory circuit includes a transistor and a capacitor, and the transistor is preferably a transistor including an oxide semiconductor in a semiconductor layer including a channel formation region (hereinafter, an OS transistor). An OS transistor has an extremely low leakage current that flows in an off state. Therefore, by utilizing the characteristics of an OS transistor that enables charge retention by being turned off, data can be stored. The configuration of the neural network circuit will be described in detail in Embodiment 3.

Another embodiment of the present invention is the recording medium that records a control program and control software, which are capable of physical property prediction by generating fingerprints with connected or parallel notation using a plurality of fingerprint types and performing machine learning.

### Embodiment 3

In this embodiment, a configuration example of a semiconductor device that can be used in the neural network circuits described in the above embodiment is described.

Note that in this specification, a semiconductor device refers to a device that can function by utilizing semiconductor characteristics. That is, a neural network circuit including a transistor that utilizes semiconductor characteristics is a semiconductor device.

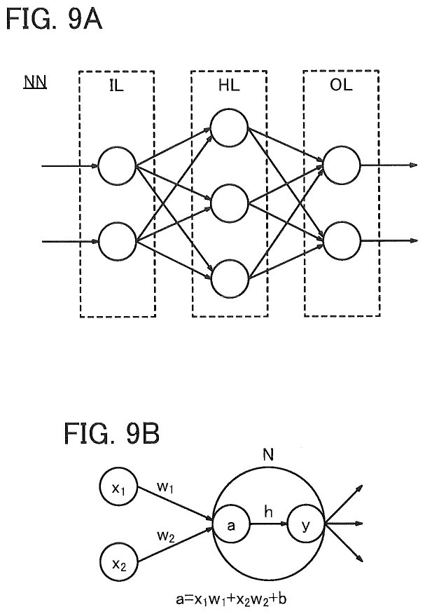

As shown in FIG. 9(A), a neural network NN can be formed of an input layer IL, an output layer OL, and a middle layer (hidden layer) HL. The input layer IL, the output layer OL, and the middle layer HL each include one or more neurons (units). Note that the middle layer HL may be composed of one layer or two or more layers. A neural network including two or more middle layers HL can also be referred to as a DNN (a deep neural network), and learning using a deep neural network can also be referred to as deep learning.

Input data is input to each neuron of the input layer IL, output signals of neurons in the previous layer or the subsequent layer are input to neurons of the middle layer HL, and output signals of neurons in the previous layer are input to neurons of the output layer OL. Note that each neuron may be connected to all the neurons in the previous and subsequent layers (full connection), or may be connected to some of the neurons.

FIG. 9(B) shows an example of an operation with the neurons. Here, a neuron N and two neurons in the previous layer which output signals to the neuron N are shown. An output x₁ of the neuron in the previous layer and an output x₂ of the neuron in the previous layer are input to the neuron N. Then, in the neuron N, a total sum x₁w₁+x₂w₂ of the product of the output x₁ and a weight w₁ (x₁w₁) and the product of the output x₂ and a weight w₂ (x₂w₂) is calculated, and then a bias b is added as necessary, so that a value a=x₁w₁+x₂w₂+b is obtained. Then, the value a is converted with an activation function h, and an output signal y=h(a) is output from the neuron N.

In this manner, the calculation by the neurons includes the calculation that sums the products of the outputs and the weights of the neurons in the previous layer, that is, the product-sum operation (x₁w₁+x₂w₂ described above). This product-sum operation may be performed using a program on software or using hardware. In the case where the product-sum operation is performed by hardware, a product-sum arithmetic circuit can be used. Either a digital circuit or an analog circuit may be used as this product-sum arithmetic circuit. In the case where an analog circuit is used as the product-sum arithmetic circuit, the circuit scale of the product-sum arithmetic circuit can be reduced, or higher processing speed and lower power consumption can be achieved by reduced frequency of access to a memory.

The product-sum arithmetic circuit may include a transistor including silicon (such as single crystal silicon) in a channel formation region (hereinafter also referred to as a Si transistor) or a transistor including an oxide semiconductor in a channel formation region (hereinafter also referred to as an OS transistor). An OS transistor is particularly suitable as a transistor included in an analog memory of the product-sum arithmetic circuit because of its extremely low off-state current. Note that the product-sum arithmetic circuit may include both a Si transistor and an OS transistor. A configuration example of a semiconductor device having a function of the product-sum arithmetic circuit will be described below.

### <Configuration Example of Semiconductor Device>

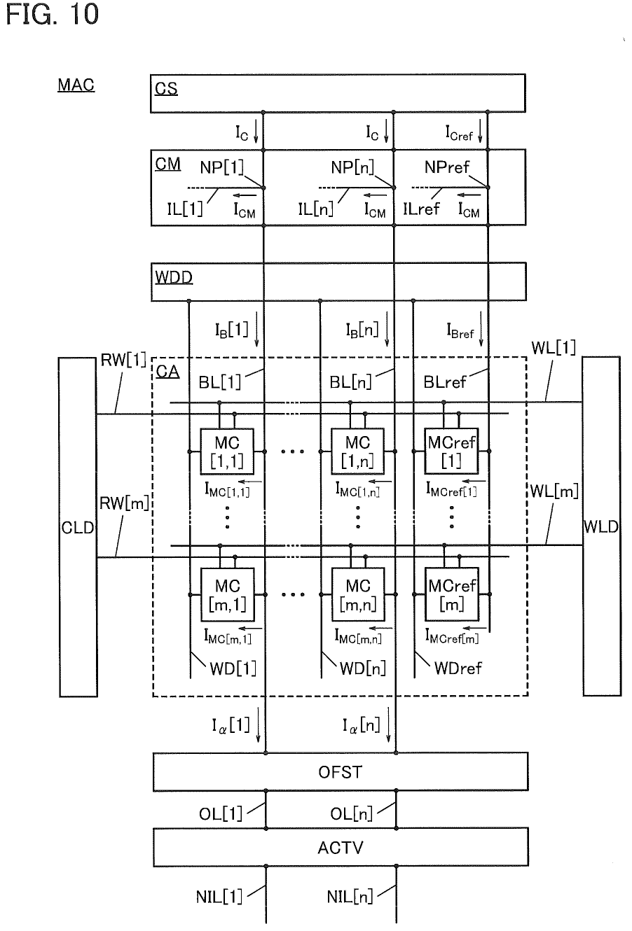

FIG. 10 illustrates a configuration example of a semiconductor device MAC having a function of performing an operation of a neural network. The semiconductor device MAC has a function of performing a product-sum operation of first data corresponding to the connection strength (weight) between the neurons and second data corresponding to input data. Note that the first data and the second data can each be analog data or multilevel data (discrete data). The semiconductor device MAC also has a function of converting data obtained by the product-sum operation with the activation function.

The semiconductor device MAC includes a cell array CA, a current source circuit CS, a current mirror circuit CM, a circuit WDD, a circuit WLD, a circuit CLD, an offset circuit OFST, and an activation function circuit ACTV.

The cell array CA includes a plurality of memory cells MC and a plurality of memory cells MCref. FIG. 10 illustrates a configuration example in which the cell array CA includes the memory cells MC in m rows and n columns (MC[1, 1] to MC[m, n]) and the m memory cells MCref (MCref[1] to MCref[m]) (m and n are integers greater than or equal to 1). The memory cells MC each have a function of storing the first data. In addition, the memory cells MCref each have a function of storing reference data used for the product-sum operation. Note that the reference data can be analog data or multilevel data.

The memory cell MC[i, j] (i is an integer greater than or equal to 1 and less than or equal to m, and j is an integer greater than or equal to 1 and less than or equal to n) is connected to a wiring WL[i], a wiring RW[i], a wiring WD[j], and a wiring BL[j]. In addition, the memory cell MCref[i] is connected to the wiring WL[i], the wiring RW[i], a wiring WDref, and a wiring BLref. Here, a current flowing between the memory cell MC[i, j] and the wiring BL[j] is denoted by I_{MC[i,f]}, and a current flowing between the memory cell MCref[i] and the wiring BLref is denoted by I_{MCref[f]}.

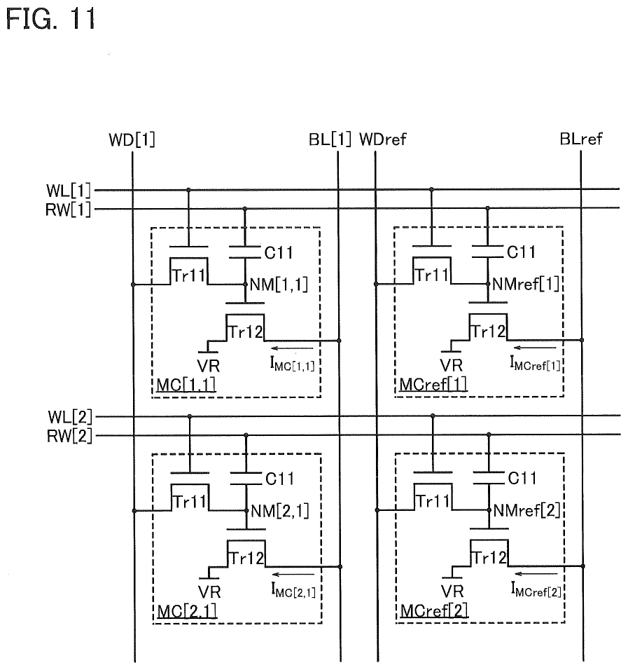

FIG. 11 illustrates a specific configuration example of the memory cells MC and the memory cells MCref. Although the memory cells MC[1, 1] and MC[2, 1] and the memory cells MCref[1] and MCref[2] are illustrated in FIG. 11 as typical examples, similar configurations can be used for other memory cells MC and memory cells MCref. The memory cells MC and the memory cells MCref each include transistors Tr11 and Tr12 and a capacitor C11. Here, the case where the transistor Tr11 and the transistor Tr12 are n-channel transistors will be described.

In the memory cell MC, a gate of the transistor Tr11 is connected to the wiring WL, one of a source and a drain is connected to a gate of the transistor Tr12 and a first electrode of the capacitor C11, and the other of the source and the drain is connected to the wiring WD. One of a source and a drain of the transistor Tr12 is connected to the wiring BL, and the other of the source and the drain is connected to a wiring VR. A second electrode of the capacitor C11 is connected to the wiring RW. The wiring VR is a wiring having a function of supplying a predetermined potential. Here, the case where a low power supply potential (e.g., a ground potential) is supplied from the wiring VR is described as an example.

A node connected to the one of the source and the drain of the transistor Tr11, the gate of the transistor Tr12, and the first electrode of the capacitor C11 is referred to as a node NM. The nodes NM in the memory cells MC[1, 1] and MC[2, 1] are referred to as nodes NM[1, 1] and NM[2, 1], respectively.

The memory cells MCref have a configuration similar to that of the memory cell MC. However, the memory cells MCref are connected to the wiring WDref instead of the wiring WD and connected to the wiring BLref instead of the wiring BL. Nodes in the memory cells MCref[1] and MCref[2] each of which is connected to the one of the source and the drain of the transistor Tr11, the gate of the transistor Tr12, and the first electrode of the capacitor C11 are referred to as nodes NMref[1] and NMref[2], respectively.

The node NM and the node NMref function as holding nodes of the memory cell MC and the memory cell MCref, respectively. The first data is held in the node NM and the reference data is held in the node NMref. Currents I_{MC[1, 1]} and I_{MC[2, 1]} from the wiring BL[1] flow to the transistors Tr12 of the memory cells MC[1, 1] and MC[2, 1], respectively. Currents I_{MCref[1]} and I_{MCref[2]} from the wiring BLref flow to the transistors Tr12 of the memory cells MCref[1] and MCref[2], respectively.

Since the transistor Tr11 has a function of holding the potential of the node NM or the node NMref, the off-state current of the transistor Tr11 is preferably low. Thus, it is preferable to use an OS transistor, which has extremely low off-state current, as the transistor Tr11. This inhibits a change in the potential of the node NM or the node NMref, so that the operation accuracy can be improved. Furthermore, operations of refreshing the potential of the node NM or the node NMref can be performed less frequently, which leads to a reduction in power consumption.

There is no particular limitation on the transistor Tr12, and for example, a Si transistor, an OS transistor, or the like can be used. In the case where an OS transistor is used as the transistor Tr12, the transistor Tr12 can be manufactured with the same manufacturing apparatus as the transistor Tr11, and accordingly manufacturing cost can be reduced. Note that the transistor Tr12 may be an n-channel transistor or a p-channel transistor.

The current source circuit CS is connected to the wirings BL[1] to BL[n] and the wiring BLref. The current source circuit CS has a function of supplying currents to the wirings BL[1] to BL[n] and the wiring BLref. Note that the value of the current supplied to the wirings BL[1] to BL[n] may be different from the value of the current supplied to the wiring BLref. Here, the current supplied from the current source circuit CS to the wirings BL[1] to BL[n] is denoted by Ic, and the current supplied from the current source circuit CS to the wiring BLref is denoted by I_{Cref}.

The current mirror circuit CM includes wirings IL[1] to IL[n] and a wiring ILref. The wirings IL[1] to IL[n] are connected to the wirings BL[1] to BL[n], respectively, and the wiring ILref is connected to the wiring BLref. Here, portions where the wirings IL[1] to IL[n] are connected to the respective wirings BL[1] to BL[n] are referred to as nodes NP[1] to NP[n]. Furthermore, a portion where the wiring ILref is connected to the wiring BLref is referred to as a node NPref.

The current mirror circuit CM has a function of making a current I_{CM} corresponding to the potential of the node NPref flow to the wiring ILref and a function of making this current I_{CM} flow also to the wirings IL[1] to IL[n]. In the example illustrated in FIG. 10, the current I_{CM} is discharged from the wiring BLref to the wiring ILref, and the current I_{CM} is discharged from the wirings BL[1] to BL[n] to the wirings IL[1] to IL[n]. Furthermore, currents flowing from the current mirror circuit CM to the cell array CA through the wirings BL[1] to BL[n] are denoted by I_{B}[1] to I_{B}[n]. Furthermore, a current flowing from the current mirror circuit CM to the cell array CA through the wiring BLref is denoted by I_{Bref}.

The circuit WDD is connected to the wirings WD[1] to WD[n] and the wiring WDref. The circuit WDD has a function of supplying a potential corresponding to the first data to be stored in the memory cells MC to the wirings WD[1] to WD[n]. The circuit WDD also has a function of supplying a potential corresponding to the reference data to be stored in the memory cell MCref to the wiring WDref. The circuit WLD is connected to wirings WL[1] to WL[m]. The circuit WLD has a function of supplying a signal for selecting the memory cell MC or the memory cell MCref to which data is to be written, to any of the wirings WL[1] to WL[m]. The circuit CLD is connected to the wirings RW[1] to RW[m]. The circuit CLD has a function of supplying a potential corresponding to the second data to the wirings RW[1] to RW[m].

The offset circuit OFST is connected to the wirings BL[1] to BL[n] and wirings OL[1] to OL[n]. The offset circuit OFST has a function of detecting the amount of currents flowing from the wirings BL[1] to BL[n] to the offset circuit OFST and/or the amount of change in the currents flowing from the wirings BL[1] to BL[n] to the offset circuit OFST. The offset circuit OFST also has a function of outputting detection results to the wirings OL[1] to OL[n]. Note that the offset circuit OFST may output currents corresponding to the detection results to the wirings OL, or may convert the currents corresponding to the detection results into voltages to output the voltages to the wirings OL. The currents flowing between the cell array CA and the offset circuit OFST are denoted by I_{α}[1] to I_{α}[n].

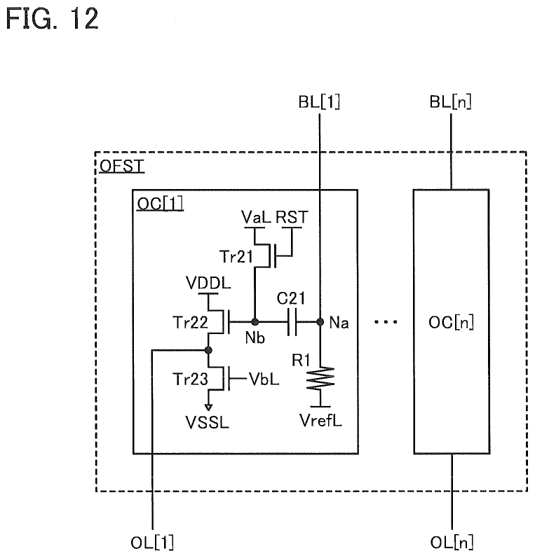

FIG. 12 illustrates a configuration example of the offset circuit OFST. The offset circuit OFST illustrated in FIG. 12 includes circuits OC[1] to OC[n]. The circuits OC[1] to OC[n] each include a transistor Tr21, a transistor Tr22, a transistor Tr23, a capacitor C21, and a resistor R1. Connection relations of the elements are illustrated in FIG. 12. Note that a node connected to a first electrode of the capacitor C21 and a first terminal of the resistor R1 is referred to as a node Na. In addition, a node connected to a second electrode of the capacitor C21, one of a source and a drain of the transistor Tr21, and a gate of the transistor Tr22 is referred to as a node Nb.

A wiring VrefL has a function of supplying a potential Vref, a wiring VaL has a function of supplying a potential Va, and a wiring VbL has a function of supplying a potential Vb. Furthermore, a wiring VDDL has a function of supplying a potential VDD, and a wiring VSSL has a function of supplying a potential VSS. Here, the case where the potential VDD is a high power supply potential and the potential VSS is a low power supply potential is described. A wiring RST has a function of supplying a potential for controlling the conduction state of the transistor Tr21. The transistor Tr22, the transistor Tr23, the wiring VDDL, the wiring VSSL, and the wiring VbL form a source follower circuit.

Next, an operation example of the circuits OC[1] to OC[n] will be described. Note that although an operation example of the circuit OC[1] is described here as a typical example, the circuits OC[2] to OC[n] can operate in a similar manner. First, when a first current flows to the wiring BL[1], the potential of the node Na becomes a potential corresponding to the first current and the resistance value of the resistor R1. At this time, the transistor Tr21 is in an on state, and thus the potential Va is supplied to the node Nb. Then, the transistor Tr21 is brought into an off state.

Next, when a second current flows to the wiring BL[1], the potential of the node Na changes to a potential corresponding to the second current and the resistance value of the resistor R1. At this time, since the transistor Tr21 is in an off state and the node Nb is in a floating state, the potential of the node Nb changes because of capacitive coupling, following the change in the potential of the node Na. Here, when the amount of change in the potential of the node Na is ΔV_{Na} and the capacitive coupling coefficient is 1, the potential of the node Nb is Va+ΔV_{Na}. When the threshold voltage of the transistor Tr22 is Vₜₕ, a potential Va+ΔV_{Na}−Vₜₕ is output from the wiring OL[1]. Here, when Va=Vₜₕ, the potential ΔV_{Na} can be output from the wiring OL[1].

The potential ΔV_{Na} is determined by the amount of change from the first current to the second current, the resistor R1, and the potential Vref. Here, since the resistor R1 and the potential Vref are known, the amount of change in the current flowing to the wiring BL can be found from the potential ΔV_{Na}.

A signal corresponding to the amount of current and/or the amount of change in the current that are/is detected by the offset circuit OFST as described above is input to the activation function circuit ACTV through the wirings OL[1] to OL[n].

The activation function circuit ACTV is connected to the wirings OL[1] to OL[n] and wirings NIL[1] to NIL[n]. The activation function circuit ACTV has a function of performing an operation for converting the signal input from the offset circuit OFST in accordance with the predefined activation function. As the activation function, for example, a sigmoid function, a tanh function, a softmax function, a ReLU function, a threshold function, or the like can be used. The signal converted by the activation function circuit ACTV is output as output data to the wirings NIL[1] to NIL[n].

### <Operation Example of Semiconductor Device>

The product-sum operation of the first data and the second data can be performed using the above semiconductor device MAC. An operation example of the semiconductor device MAC at the time of performing the product-sum operation is described below.

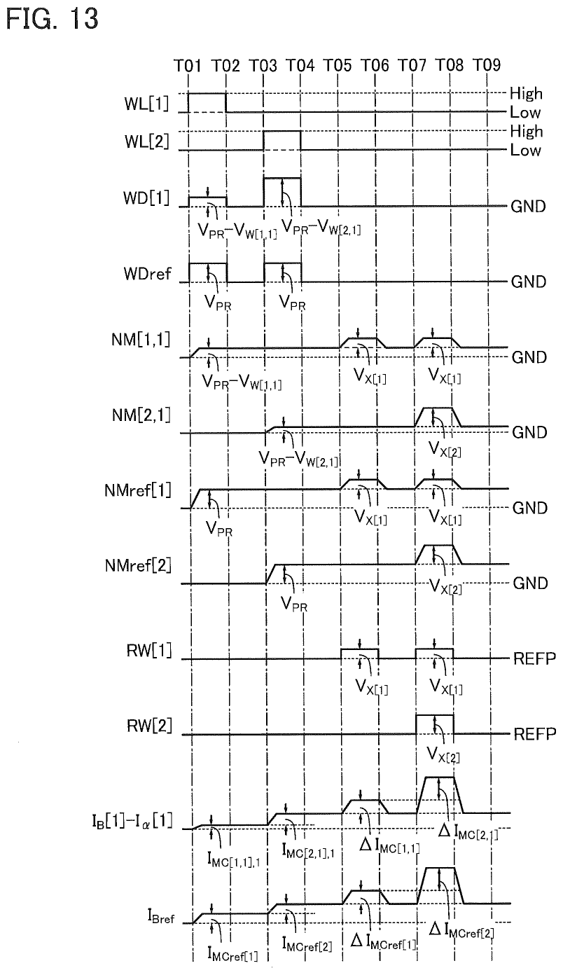

FIG. 13 illustrates a timing chart of the operation example of the semiconductor device MAC. FIG. 13 shows changes in the potentials of the wiring WL[1], the wiring WL[2], the wiring WD[1], the wiring WDref, the node NM[1, 1], the node NM[2, 1], the node NMref[1], the node NMref[2], the wiring RW[1], and the wiring RW[2] in FIG. 11 and changes in the values of a current I_{B}[1]-I_{α}[1] and the current I_{Bref}. The current I_{B}[1]-I_{α}[1] corresponds to the sum total of the currents flowing from the wiring BL[1] to the memory cells MC[1, 1] and MC[2, 1].

Although an operation is described with a focus on the memory cells MC[1, 1] and MC[2, 1] and the memory cells MCref[1] and MCref[2] illustrated in FIG. 11 as a typical example, the other memory cells MC and the other memory cells MCref can be operated in a similar manner.

### [Storage of First Data]

First, from Time T01 to T02, the potential of the wiring WL[1] becomes a high level (High), the potential of the wiring WD[1] becomes a potential greater than a ground potential (GND) by V_{PR}−V_{W[1,1]}, and the potential of the wiring WDref becomes a potential greater than the ground potential by V_{PR}. The potentials of the wiring RW[1] and the wiring RW[2] become reference potentials (REFP). Note that the potential V_{W[1, 1]} is a potential corresponding to the first data stored in the memory cell MC[1, 1]. The potential V_{PR} is a potential corresponding to the reference data. Thus, the transistors Tr11 included in the memory cell MC[1, 1] and the memory cell MCref[1] are brought into on states, and the potential of the node NM[1, 1] becomes V_{PR}−V_{W[1, 1]} and the potential of the node NMref[1] becomes VPR.

In this case, a current I_{MC[1, 1], 0} flowing from the wiring BL[1] to the transistor Tr12 in the memory cell MC[1, 1] can be expressed by the following formula. Here, k is a constant determined by the channel length, the channel width, the mobility, the capacitance of a gate insulating film, and the like of the transistor Tr12. Furthermore, Vₜₕ is the threshold voltage of the transistor Tr12.

I_{MC[1, 1], 0}=k(V_{PR}−V_{W[1, 1]}−Vₜₕ)² (E1)

Furthermore, a current I_{MCref[1], 0} flowing from the wiring BLref to the transistor Tr12 in the memory cell MCref[1] can be expressed by the following formula.

I_{MCref[1], 0}=k(V_{PR}−Vₜₕ)² (E2)

Next, from Time T02 to T03, the potential of the wiring WL[1] becomes a low level (Low). Consequently, the transistors Tr11 included in the memory cell MC[1, 1] and the memory cell MCref[1] are brought into off states, and the potentials of the node NM[1, 1] and the node NMref[1] are retained.

As described above, an OS transistor is preferably used as the transistor Tr11. This can suppress the leakage current of the transistor Tr11, so that the potentials of the node NM[1, 1] and the node NMref[1] can be retained accurately.

Next, from Time T03 to T04, the potential of the wiring WL[2] becomes the high level, the potential of the wiring WD[1] becomes a potential greater than the ground potential by V_{PR}−V_{W[2, 1]}, and the potential of the wiring WDref becomes a potential greater than the ground potential by V_{PR}. Note that the potential V_{W[2, 1]} is a potential corresponding to the first data stored in the memory cell MC[2, 1]. Thus, the transistors Tr11 included in the memory cell MC[2, 1] and the memory cell MCref[2] are brought into on states, and the potential of the node NM[1, 1] becomes V_{PR}−V_{W[2, 1]} and the potential of the node NMref[1] becomes VPR.

In this case, a current I_{MC[2, 1], 0} flowing from the wiring BL[1] to the transistor Tr12 in the memory cell MC[2, 1] can be expressed by the following formula.

I_{MC[2, 1], 0}=k(V_{PR}−V_{W[2, 1]}−Vₜₕ)² (E3)

Furthermore, a current I_{MCref[2], 0} flowing from the wiring BLref to the transistor Tr12 in the memory cell MCref[2] can be expressed by the following formula.

I_{MCref[2], 0}=k(V_{PR}−Vₜₕ)² (E4)

Next, from Time T04 to T05, the potential of the wiring WL[2] becomes the low level. Consequently, the transistors Tr11 included in the memory cell MC[2, 1] and the memory cell MCref[2] are brought into off states, and the potentials of the node NM[2, 1] and the node NMref[2] are retained.

Through the above operation, the first data is stored in the memory cells MC[1, 1] and MC[2, 1], and the reference data is stored in the memory cells MCref[1] and MCref[2].

Here, currents flowing through the wiring BL[1] and the wiring BLref from Time T04 to T05 are considered. A current is supplied from the current source circuit CS to the wiring BLref. The current flowing through the wiring BLref is discharged to the current mirror circuit CM and the memory cells MCref[1] and MCref[2]. The following formula holds where I_{Cref} is the current supplied from the current source circuit CS to the wiring BLref and I_{CM, 0} is the current discharged from the wiring BLref to the current mirror circuit CM.

I_{Cref}−I_{CM, 0}=I_{MCref[1], 0}+I_{MCref[2], 0} (E5)

A current from the current source circuit CS is supplied to the wiring BL[1]. The current flowing through the wiring BL[1] is discharged to the current mirror circuit CM and the memory cells MC[1, 1] and MC[2, 1]. Furthermore, the current flows from the wiring BL[1] to the offset circuit OFST. The following formula holds where I_{C, 0} is the current supplied from the current source circuit CS to the wiring BL[1] and I_{α, 0} is the current flowing from the wiring BL[1] to the offset circuit OFST.

I_{C}−I_{CM, 0}=I_{MC[1, 1], 0}+I_{MC[2, 1], 0}+I_{α, 0} (E6)

### [Product-Sum Operation of First Data and Second Data]

Next, from Time T05 to T06, the potential of the wiring RW[1] becomes a potential greater than the reference potential by V_{X[1]}. At this time, the potential V_{X[1]} is supplied to the capacitor C11 in each of the memory cell MC[1, 1] and the memory cell MCref[1], so that the potential of the gate of the transistor Tr12 is increased because of capacitive coupling. Note that the potential V_{X[1]} is a potential corresponding to the second data supplied to the memory cell MC[1, 1] and the memory cell MCref[1].

The amount of change in the potential of the gate of the transistor Tr12 corresponds to the value obtained by multiplying the amount of change in the potential of the wiring RW by a capacitive coupling coefficient determined by the memory cell configuration. The capacitive coupling coefficient is calculated using the capacitance of the capacitor C11, the gate capacitance of the transistor Tr12, the parasitic capacitance, and the like. In the following description, for convenience, the amount of change in the potential of the wiring RW is equal to the amount of change in the potential of the gate of the transistor Tr12, that is, the capacitive coupling coefficient is 1. In practice, the potential V_{X} can be determined in consideration of the capacitive coupling coefficient.

When the potential V_{X[1]} is supplied to the capacitors C11 in the memory cell MC[1] and the memory cell MCref[1], the potentials of the node NM[1] and the node NMref[1] each increase by V_{X[1]}.

Here, a current I_{MC[1, 1], 1} flowing from the wiring BL[1] to the transistor Tr12 in the memory cell MC[1, 1] from Time T05 to T06 can be expressed by the following formula.

I_{MC[1, 1], 1}=k(V_{PR}−V_{W[1, 1]}+V_{X[1]}−Vₜₕ)² (E7)

That is, when the potential V_{X[1]} is supplied to the wiring RW[1], the current flowing from the wiring BL[1] to the transistor Tr12 in the memory cell MC[1, 1] increases by ΔI_{MC[1, 1]}=I_{MC[1, 1], 1}−I_{MC[1, 1], 0}.

A current I_{MCref[1], 1} flowing from the wiring BLref to the transistor Tr12 in the memory cell MCref[1] from Time T05 to T06 can be expressed by the following formula.

I_{MCref[1], 1}=k(V_{PR}+V_{X[1]}−Vₜₕ)² (E8)

That is, when the potential V_{X[1]} is supplied to the wiring RW[1], the current flowing from the wiring BLref to the transistor Tr12 in the memory cell MCref[1] increases by ΔI_{MCref}[1]=I_{MCref[1], 1}−I_{MCref[1], 0}.

Furthermore, currents flowing through the wiring BL[1] and the wiring BLref are considered. The current I_{Cref} is supplied from the current source circuit CS to the wiring BLref. The current flowing through the wiring BLref is discharged to the current mirror circuit CM and the memory cells MCref[1] and MCref[2]. The following formula holds where I_{CM, 1} is the current discharged from the wiring BLref to the current mirror circuit CM.

I_{Cref}−I_{CM, 1}=I_{MCref[1], 1}+I_{MCref[2], 0} (E9)

The current I_{C} from the current source circuit CS is supplied to the wiring BL[1]. The current flowing through the wiring BL[1] is discharged to the current mirror circuit CM and the memory cells MC[1, 1] and MC[2, 1]. Furthermore, the current flows from the wiring BL[1] to the offset circuit OFST. The following formula holds where I_{α, 1} is the current flowing from the wiring BL[1] to the offset circuit OFST.

I_{C}−I_{CM, 1}=I_{MC[1, 1], 1}+I_{MC[2, 1], 1}+I_{α, 1} (E10)

In addition, from the formula (E1) to the formula (E10), a difference between the current I_{α, 0} and the current I_{α, 1} (differential current ΔI_{α}) can be expressed by the following formula.

ΔI_{α}=I_{α, 0}−I_{α, 1}=2 kV_{W[1, 1]}V_{X[1]} (E11)

Thus, the differential current ΔI_{α} is a value corresponding to the product of the potentials V_{W[1, 1]} and V_{X[1]}.

After that, from Time T06 to T07, the potential of the wiring RW[1] becomes the ground potential, and the potentials of the node NM[1, 1] and the node NMref[1] become similar to those from Time T04 to T05.

Next, from Time T07 to T08, the potential of the wiring RW[1] becomes a potential greater than the reference potential by V_{X[1]}, and the potential of the wiring RW[2] becomes a potential greater than the reference potential by V_{X[2]}. Accordingly, the potential V_{X[1]} is supplied to the capacitor C11 in each of the memory cell MC[1, 1] and the memory cell MCref[1], and the potentials of the node NM[1, 1] and the node NMref[1] each increase by V_{X[1]} because of capacitive coupling. Furthermore, the potential V_{X[2]} is supplied to the capacitor C11 in each of the memory cell MC[2, 1] and the memory cell MCref[2], and the potentials of the node NM[2, 1] and the node NMref[2] each increase by V_{X[2]} because of capacitive coupling.

Here, a current I_{MC[2, 1], 1} flowing from the wiring BL[1] to the transistor Tr12 in the memory cell MC[2, 1] from Time T07 to T08 can be expressed by the following formula.

I_{MC[2, 1], 1}=k(V_{PR}−V_{W[2, 1]}+V_{X[2]}−Vₜₕ)² (E12)

That is, when the potential V_{X[2]} is supplied to the wiring RW[2], the current flowing from the wiring BL[1] to the transistor Tr12 in the memory cell MC[2, 1] increases by ΔI_{MC[2, 1]}=I_{MC[2, 1], 1}−I_{MC[2, 1], 0}.

A current I_{MCref[2], 1} flowing from the wiring BLref to the transistor Tr12 in the memory cell MCref[2] from Time T05 to T06 can be expressed by the following formula.

I_{MCref[2], 1}=k(V_{PR}+V_{x[2]}−Vₜₕ)² (E13)

That is, when the potential V_{X[2]} is supplied to the wiring RW[2], the current flowing from the wiring BLref to the transistor Tr12 in the memory cell MCref[2] increases by ΔI_{MCref[2]}=I_{MCref[2], 0}.

Furthermore, currents flowing through the wiring BL[1] and the wiring BLref are considered. The current I_{Cref} is supplied from the current source circuit CS to the wiring BLref. The current flowing through the wiring BLref is discharged to the current mirror circuit CM and the memory cells MCref[1] and MCref[2]. The following formula holds where I_{CM, 2} is the current discharged from the wiring BLref to the current mirror circuit CM.

I_{Cref}−I_{CM, 2}=I_{MCref[1], 1}+I_{MCref[2], 1} (E14)

The current I_{C} from the current source circuit CS is supplied to the wiring BL[1]. The current flowing through the wiring BL[1] is discharged to the current mirror circuit CM and the memory cells MC[1, 1] and MC[2, 1]. Furthermore, the current flows from the wiring BL[1] to the offset circuit OFST. The following formula holds where I_{α, 2} is the current flowing from the wiring BL[1] to the offset circuit OF ST.

I_{C}−I_{CM, 2}=I_{MC[1, 1], 1}+I_{MC[2, 1], 1}+I_{α, 2} (E15)

In addition, from the formula (E1) to the formula (E8) and the formula (E12) to the formula (E15), a difference between the current I_{α, 0} and the current I_{α, 2} (differential current ΔIa) can be expressed by the following formula.

ΔI_{α}=I_{α, 0}−I_{α, 2}=2k(V_{W[1, 1]}V_{X[1]}+V_{W[2, 1]}V_{X[2]}) E16)

Thus, the differential current ΔI_{α} is a value corresponding to the sum of the product of the potential V_{W[1, 1]} and the potential V_{X[1]} and the product of the potential V_{W[2, 1]} and the potential V_{X[2]}.

After that, from Time T08 to T09, the potentials of the wirings RW[1] and RW[2] become the ground potential, and the potentials of the nodes NM[1, 1] and NM[2, 1] and the nodes NMref[1] and NMref[2] become similar to those from Time T04 to T05.

As represented by the formula (E9) and the formula (E16), the differential current ΔI_{α} input to the offset circuit OFST is a value corresponding to the sum of the products of the potentials V_{X} corresponding to the first data (weight) and the potentials V_{W} corresponding to the second data (input data). In other words, measurement of the differential current ΔI_{α} with the offset circuit OFST gives the result of the product-sum operation of the first data and the second data.

Note that although the memory cells MC[1, 1] and MC[2, 1] and the memory cells MCref[1] and MCref[2] are particularly focused on in the above description, the number of the memory cells MC and the memory cells MCref can be freely set. In the case where the number m of rows of the memory cells MC and the memory cells MCref is an arbitrary number, the differential current ΔI_{α} can be expressed by the following formula.

ΔI_{α}=2kΣᵢV_{W[i, 1]}V_{X[i]} (E17)

When the number n of columns of the memory cells MC and the memory cells MCref is increased, the number of product-sum operations executed in parallel can be increased.

The product-sum operation of the first data and the second data can be performed using the semiconductor device MAC as described above. Note that the use of the configuration of the memory cells MC and the memory cells MCref in FIG. 11 allows the product-sum operation circuit to be formed of fewer transistors. Accordingly, the circuit scale of the semiconductor device MAC can be reduced.

In the case where the semiconductor device MAC is used for the operation in the neural network, the number m of rows of the memory cells MC can correspond to the number of pieces of input data supplied to one neuron and the number n of columns of the memory cells MC can correspond to the number of neurons. For example, the case where a product-sum operation using the semiconductor device MAC is performed in the middle layer HL in FIG. 9(A) is considered. In this case, the number m of rows of the memory cells MC can be set to the number of pieces of input data supplied from the input layer IL (the number of neurons in the input layer IL), and the number n of columns of the memory cells MC can be set to the number of neurons in the middle layer HL.

Note that there is no particular limitation on the structure of the neural network for which the semiconductor device MAC is used. For example, the semiconductor device MAC can also be used for a convolutional neural network (CNN), a recurrent neural network (RNN), an autoencoder, a Boltzmann machine (including a restricted Boltzmann machine), or the like.

The product-sum operation in the neural network can be performed using the semiconductor device MAC as described above. Furthermore, the memory cells MC and the memory cells MCref illustrated in FIG. 11 are used for the cell array CA, whereby an integrated circuit IC with improved operation accuracy, lower power consumption, or a reduced circuit scale can be provided.

### EXAMPLE 1

In this example, an example of prediction of a physical property of an organic compound is described in detail. In this example, the T1 level was selected as the physical property value to be predicted in connection with the molecular structure of an organic compound. The value of the T1 level used for learning is a value found from an emission peak wavelength on the short wavelength side of a phosphorescence spectrum obtained by low-temperature PL measurement. The total number of data was 420, 380 of which were used for learning and 40 of which were used for testing, whereby the validity of the prediction model was evaluated.

For mathematization of the molecular structure, RDKit, which is an open-source cheminformatics toolkit, was employed. In the RDKit, the SMILES notation of the molecular structure can be converted into mathematical expression data by a fingerprinting method. As the fingerprinting method, a Circular type and an Atom pair type were used.

As input values for physical property prediction, a mathematical expression notated by the Circular type alone, a mathematical expression notated by the Atom pair type alone, and further a mathematical expression in which both were connected were used. A radius was specified as 4 in the Circular type, and a path length was specified as 30 in the Atom pair type. The bit length of each fingerprint was set to 2048. The radius in the Circular type or the path length in the Atom pair type is the number of bonded elements counted starting from a certain element as 0.

In the case of notation by the Circular type alone, mathematical expressions of two pairs among the 420 kinds of organic compounds were the same. By contrast, in the cases of notation by the Atom pair type alone or notation by the connected Circular type and Atom pair type, it was confirmed that the mathematical expressions of the different organic compounds were all different, not the same.

As a method of machine learning, a neural network was used. Python was used as a programming language and Chainer was used as a framework of machine learning. In the structure of the neural network, two hidden layers were used. The number of neurons in each layer was as follows: 2048 (the number of bits for the Circular type alone or the Atom pair type alone) or 4096 (the numbers of bits for the connected Circular type and Atom pair type) in an input layer; 500 in a first hidden layer and a second hidden layer; and 1 in an output layer. A ReLU function was used as an activation function of the hidden layers.

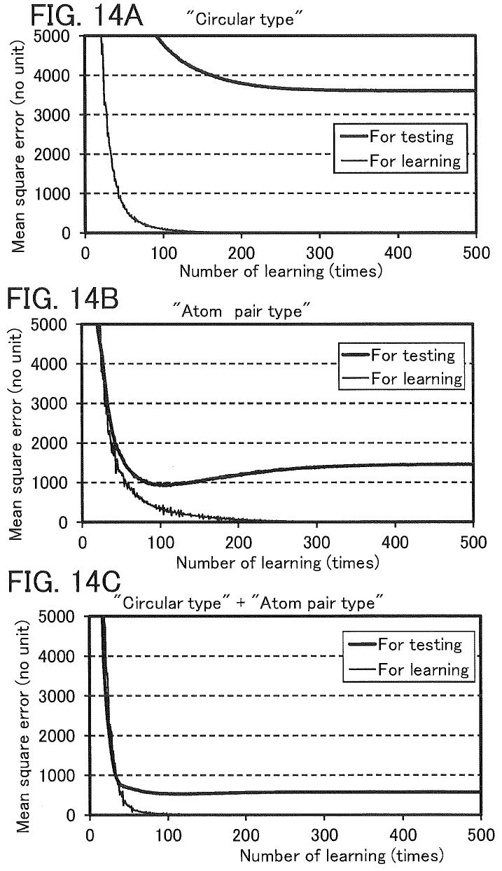

Machine learning was performed under the above conditions, and a change in mean square error related to data for learning and data for testing were determined until learning was performed 500 times. FIG. 14 shows the results. FIG. 14(A) shows results of learning using the mathematical expressions notated by the Circular type alone. FIG. 14(B) shows results of learning using the mathematical expressions notated by the Atom pair type alone. FIG. 14(C) shows results of learning using the mathematical expressions notated by the connected Circular type and Atom pair type.

The above-described results show that, when the mathematical expressions connecting notations by Circular type and Atom pair type fingerprinting methods were used, the mean square error of data for testing decreased and the prediction accuracy of the T1 level improved than when each alone was used.

As described above, substructures differing between fingerprint types are generated, and information about the presence or absence of these substructures can complement information related to the whole molecular structure. This indicates that a method in which a plurality of fingerprinting methods of different types are used to describe a molecular structure is effective for physical property prediction using machine learning.

As described above, in the case where there are different compounds whose notations by one fingerprinting method are the same, another fingerprint is connected, so that the generated mathematical expressions can be made different more easily. Since the generated mathematical expressions are less likely to be the same and a difference between compounds can be expressed with as few bits as possible, combining two or more kinds of fingerprints is preferable and more effective than when only one kind of fingerprint type is used and the number of bits is increased until there disappear compounds with the same notation. As a result, a computational load during machine learning can be made small.

### REFERENCE NUMERALS

T01 to T02: time, T02 to T03: time, T03 to T04: time, T04 to T05: time, T05 to T06: time, T06 to T07: time, T07 to T08: time, T08 to T09: time, Tr11: transistor, Tr12: transistor, Tr21: transistor, Tr22: transistor, Tr23: transistor, 20: information terminal, 21: input portion, 22: arithmetic portion, 25: output portion, 30: data server

1. A method of predicting a physical property, comprising:
the step of learning a correlation between a molecular structure and a physical property of an organic compound; and
the step of predicting a target physical property from a molecular structure of an object substance on the basis of a result of learning,
wherein a plurality of kinds of fingerprinting methods are used at the same time as notation methods of the molecular structure of the organic compound.

2. The method of predicting a physical property according to claim 1,
wherein two kinds of fingerprinting methods are used as the plurality of kinds of fingerprinting methods.

3. The method of predicting a physical property according to claim 1,
wherein three kinds of fingerprinting methods are used as the plurality of kinds of fingerprinting methods.

4. The method of predicting a physical property according to claim 1,
wherein the plurality of kinds of fingerprinting methods comprise at least any one of an Atom pair type, a Circular type, a Substructure key type, and a Path-based type.

5. The method of predicting a physical property according to claim 1,
wherein the plurality of kinds of fingerprinting methods are selected from an Atom pair type, a Circular type, a Substructure key type, and a Path-based type.

6. The method of predicting a physical property according to claim 2, wherein the two kinds of fingerprinting methods comprise an Atom pair type and a Circular type.

7. The method of predicting a physical property according to claim 2, wherein the two kinds of fingerprinting methods comprise a Circular type and a Substructure key type.

8. The method of predicting a physical property according to claim 2, wherein the two kinds of fingerprinting methods comprise a Circular type and a Path-based type.

9. The method of predicting a physical property according to claim 2, wherein the two kinds of fingerprinting methods comprise an Atom pair type and a Substructure key type.

10. The method of predicting a physical property according to claim 2, wherein the two kinds of fingerprinting methods comprise an Atom pair type and a Path-based type.

11. The method of predicting a physical property according to claim 3, wherein the three kinds of fingerprinting methods comprise an Atom pair type, a Substructure key type, and a Circular type.

12. The method of predicting a physical property according to claim 1,
wherein r is greater than or equal to 3 when a Circular type is used for one of the plurality of kinds of fingerprinting methods, and
wherein r is the number of bonded elements counted starting from a certain element as 0.

13. The method of predicting a physical property according to claim 12,
wherein r is greater than or equal to 5 in the Circular type.

14. The method of predicting a physical property according to claim 1,
wherein notations of all organic compounds are different when molecular structures of the organic compounds to be learned are notated using at least one of the plurality of kinds of fingerprinting methods.

15. The method of predicting a physical property according to claim 1,
wherein at least one of the plurality of kinds of fingerprinting methods is capable of expressing information about a structure featuring a physical property to be predicted.

16. The method of predicting a physical property according to claim 1,
wherein at least one of the plurality of kinds of fingerprinting methods is capable of expressing at least one of a substituent, a substitution position of the substituent, a functional group, the number of elements, kinds of elements, valences of elements, a bond order, and an atomic coordinate.

17. The method of predicting a physical property according to claim 1,
wherein the physical property is any one or more of an emission spectrum; a half width; emission energy; an excitation spectrum; an absorption spectrum; a transmission spectrum; a reflectance spectrum; a molar absorption coefficient; excitation energy; a transient emission lifetime; a transient absorption lifetime; an S1 level; a T1 level; an Sn level; a Tn level; a Stokes shift value; an emission quantum yield; oscillator strength; an oxidation potential; a reduction potential; a HOMO level; a LUMO level; a glass transition point; a melting point; a crystallization temperature; a decomposition temperature; a boiling point; a sublimation temperature; carrier mobility; a refractive index; an orientation parameter; a mass-to-charge ratio; a spectrum, a chemical shift and the number of the elements, or a coupling constant in an NMR measurement; and a spectrum, a g-factor, a D value, or an E value in an ESR measurement.

18. A system of predicting a physical property, comprising:
an input means;
a data server;
a learning means configured to learn a correlation between a molecular structure and a physical property of an organic compound, the molecular structure and the physical property being stored in the data server;
a means configured to predict a target physical property value on the basis of a result of learning from a molecular structure of an object substance input from the input means; and
an output means configured to output the predicted physical property value,
wherein a plurality of kinds of fingerprinting methods are used at the same time as notation methods of the molecular structure of the organic compound.

19. The system of predicting a physical property according to claim 18,
wherein two kinds of fingerprinting methods are used as the plurality of kinds of fingerprinting methods.

20. The system of predicting a physical property according to claim 18,
wherein three kinds of fingerprinting methods are used as the plurality of kinds of fingerprinting methods.

21. The system of predicting a physical property according to claim 18,
wherein the plurality of kinds of fingerprinting methods comprise at least any one of an Atom pair type, a Circular type, a Substructure key type, and a Path-based type.

22. The system of predicting a physical property according to claim 18,
wherein the plurality of kinds of fingerprinting methods are selected from an Atom pair type, a Circular type, a Substructure key type, and a Path-based type.

23. The system of predicting a physical property according to claim 19,
wherein the two kinds of fingerprinting methods comprise an Atom pair type and a Circular type.

24. The system of predicting a physical property according to claim 19,
wherein the two kinds of fingerprinting methods comprise a Circular type and a Substructure key type.

12. The system of predicting a physical property according to claim 19,
wherein the two kinds of fingerprinting methods comprise a Circular type and a Path-based type.

26. The system of predicting a physical property according to claim 19,
wherein the two kinds of fingerprinting methods comprise an Atom pair type and a Substructure key type.

27. The system of predicting a physical property according to claim 19,
wherein the two kinds of fingerprinting methods comprise an Atom pair type and a Path-based type.

28. The system of predicting a physical property according to claim 20,
wherein the three kinds of fingerprinting methods comprise an Atom pair type, a Substructure key type, and a Circular type.

29. The system of predicting a physical property according to claim 18,
wherein r is greater than or equal to 3 when a Circular type is used for one of the plurality of kinds of fingerprinting methods, and
wherein r is the number of bonded elements counted starting from a certain element as 0.

30. The system of predicting a physical property according to claim 29,
wherein r is greater than or equal to 5 in the Circular type.

31. The system of predicting a physical property according to claim 18,
wherein notations of all organic compounds are different when molecular structures of the organic compounds to be learned are notated using at least one of the plurality of kinds of fingerprinting methods.

32. The system of predicting a physical property according to claim 18,
wherein at least one of the plurality of kinds of fingerprinting methods is capable of expressing information about a structure featuring a physical property to be predicted.

33. The system of predicting a physical property according to claim 18,
wherein at least one of the plurality of kinds of fingerprinting methods is capable of expressing at least one of a substituent, a substitution position of the substituent, a functional group, the number of elements, kinds of elements, valences of elements, a bond order, and an atomic coordinate.

34. The system of predicting a physical property according to claim 18,
wherein the physical property is any one or more of an emission spectrum; a half width; emission energy; an excitation spectrum; an absorption spectrum; a transmission spectrum; a reflectance spectrum; a molar absorption coefficient; excitation energy; a transient emission lifetime; a transient absorption lifetime; an S1 level; a T1 level; an Sn level; a Tn level; a Stokes shift value; an emission quantum yield; oscillator strength; an oxidation potential; a reduction potential; a HOMO level; a LUMO level; a glass transition point; a melting point; a crystallization temperature; a decomposition temperature; a boiling point; a sublimation temperature; carrier mobility; a refractive index; an orientation parameter; a mass-to-charge ratio; a spectrum, a chemical shift and the number of the elements, or a coupling constant in an NMR measurement; and a spectrum, a g-factor, a D value, or an E value in an ESR measurement.
